(19) United States
(12) Patent Application Publication
(10) Pub. No.: US 2024/0002945 A1
(43) Pub. Date: Jan. 4, 2024

(54) GRAPHENE-BASED BIOSENSOR AND DETECTION METHOD USING SAME

(71) Applicant: KOREA INSTITUTE OF SCIENCE AND TECHNOLOGY, Seoul (KR)

(72) Inventors: Kwan Hyi LEE, Seoul (KR); Youngdo JEONG, Seoul (KR); Ho Jun KIM, Seoul (KR); Sungwook PARK, Seoul (KR); Seongchan KIM, Seoul (KR)

(73) Assignee: KOREA INSTITUTE OF SCIENCE AND TECHNOLOGY, Seoul (KR)

(21) Appl. No.: 18/038,454

(22) PCT Filed: Nov. 18, 2021
(86) PCT No.: PCT/KR2021/017031

§ 371 Date: May 24, 2023

(30) Foreign Application Priority Data
Nov. 24, 2020 (KR) 10-2020-0159099
Nov. 12, 2021 (KR) 10-2021-0156070

(51) Int. Cl. C12Q 1/6886; C12Q 1/6837

(52) U.S. Cl. CPC C12Q 1/6886; C12Q 1/6837; C12Q 2600/156; C12Q 2600/178

(57) ABSTRACT

The present disclosure relates to a biosensor with surface-modified with reduced graphene oxide and a detection method using the same. The biosensor according to an aspect can effectively detect a target material in a urine sample, and thus, the use of the biosensor has an advantage in that prostate cancer can be diagnosed in a non-invasive, fast, and accurate manner.

### TECHNICAL FIELD

The present disclosure relates to a biosensor with surface-modified with reduced graphene oxide and a detection method using the same.

### BACKGROUND ART

Research on diagnosis of diseases using urine began to make a mark in earnest as a urinalysis test strip that can test sugar and protein was developed in 1958. Since then, research has been conducted on a system for diagnosis of more diseases using urine and a test method using the system. A technology capable of diagnosing various diseases including prostate cancer using urine should enable a more accurate diagnosis through comfortable condition of a patient by minimizing a patient's stress on diagnosis and examination. In addition, such a technology should be able to repeatedly and continuously monitor diseases without laying a mental or physical burden on the elderly as well as children.

Among diseases that can be diagnosed through urine, prostate cancer is the fourth most common cancer, and in males, is the second most common cancer which accounts for about 15% of male cancers. In addition, in terms of mortality, prostate cancer ranks fifth among all cancers, and recorded about 307,000 deaths in 2012. Early diagnosis technology for prostate cancer currently in use is to detect a prostate specific antigen (PSA) after a blood sample is subjected to a sample treatment process. The diagnosis of prostate cancer is determined by whether the PSA concentration exceeds 4 ng/mL. However, due to frequent cases of the diagnosis of prostate cancer even below the reference value, baseline, it is difficult to confirm the cancer by single diagnostics. In addition, a biopsy is essentially accompanied for cancer-suspected patients. However, not only is there a total of 2/3 chance of a false-positive result through examinations, but also such methods cause pain to the patients. Also, appropriate treatment according to tumor stage is not applied when misdiagnosed, giving the patients a heavy burden. Current prostate cancer diagnosis technology is mainly an invasive way, and in this regard, the patients are highly repulsed and unpleasant for repeated sample collection and tests. Also, the incidence of new secondary risks such as infection is high.

Therefore, there is a need for a device/sensor and diagnostic technology that are capable of diagnosing and monitoring diseases in urine in a non-invasive manner.

### DISCLOSURE

### Technical Problem

An aspect provides a biosensor including a reaction unit in which an interaction occurs physically or chemically with a target material in a sample, wherein the reaction unit includes reduced graphene oxide (rGO).

Another aspect provides a method of detecting a target material, the method including: contacting the sample with the biosensor; and observing an electrical conductivity change in the biosensor.

Another aspect provides a method of providing information on diagnosis of prostate cancer, the method including measuring an expression level of a prostate cancer marker by using the biosensor, from a biological sample isolated from a subject.

### Technical Solution

An aspect provides a biosensor including a reaction unit (sensing part) in which an interaction occurs physically or chemically with a target material in a sample, wherein the reaction unit includes reduced graphene oxide (rGO).

The term “biosensor” as used herein is configured to measure the presence or absence and amount of a specific biological material, and is a device that converts a physical or chemical change caused by a selective interaction between a biological element and an analyte into a recognizable optical or electrical signal. Applications of such a biosensor include use for the analysis of pollutants in the environmental field, use for the detection of biochemical weapons of mass destruction in the military field, and use for the detection of hazardous substances or spoilage-promoting substances in the food field. However, the biosensor is particularly in the spotlight for the purpose of early diagnosis of disease or biomaterials in clinical diagnosis and medical fields.

The interaction occurring physically or chemically with the target material is not particularly limited, but generally refers to an action caused by intermolecular forces resulting from at least one of a covalent bond, a hydrophobic bond, a hydrogen bond, a van der Waals bond, and a bond by electrostatic force. The covalent bond may include a coordinate bond and a dipole bond. Also, the bond by electrostatic force may include electrical repulsion in addition to electrostatic coupling. The interaction may also include a binding reaction caused by such an action above, a synthesis reaction, and a decomposition reaction. Examples of the interaction are association and dissociation between an antigen and an antibody, association and dissociation between a protein receptor and a ligand, association and dissociation between an adhesive molecule and a counterpart thereof, association and dissociation between an enzyme and a substrate, association and dissociation between an apoenzyme and a coenzyme, association and dissociation between a hexane and a protein binding thereto, association and dissociation between a hexane and a hexane, association and dissociation between proteins in the information delivery system, association and dissociation between a glycoprotein and a protein, association and dissociation between a sugar chain and a protein, association and dissociation between a protein and either of a cell and a biological tissue, association and dissociation between a low molecular weight compound and either of a cell and a biological tissue, and an interaction between an ion-sensitive material.

The reaction unit may include: a substrate; an electrode formed on the substrate; and an rGO layer formed on the electrode.

The term “graphene” as used herein refers, as one of carbon allotropes, a material that constitutes a two-dimensional planar crystal structure in the shape of a widely spread hexagonal honeycomb in which carbon atoms are at apexes of the hexagon (sp2 bonds). Graphene is a film of only one atom thick, which exists in a stable structure.

The term “graphene oxide (GO)” as used herein refers to graphene having a structure in which various (oxygen-containing) functional groups (—OH, —COOH, —C═O, —CHO, or the like) oxidized on graphene or at the edge of graphene are bonded, and is a single-layered material or a material having several to tens of layers. The GO may be obtained by a method known to those skilled in the art. For example, the GO may be obtained by first cutting graphite by applying mechanical/thermal energy thereto and granulating the resultant according to a mechanical and chemical exfoliation method.

The term “reduced graphene oxide (rGO)” as used herein refers to graphene oxide with reduced oxygen ratio through a reduction process.

The rGO may exist in various forms. For example, the rGO may be in the form of a nanofilm, a nanosheet, a nanowire, a nanorod, nanotube, pulverized nanowire, nanotetrapod, tripod, bipod, nanocrystal, nanodot, quantum wire, or nanoparticle. In detail, the rGO may be in the form of a nanosheet.

The reaction unit may include an rGO layer, and may include one or more rGO sheets.

In an embodiment, the rGO layer (nanosheet) may be bonded to the substrate of the reaction unit or the electrode formed on the substrate. In detail, a surface of the electrode may be modified and functionalized with rGO.

The reaction unit may be configured for one-time use. For example, the substrate may be a material selected from the group consisting of silicon, glass, metal, plastic, and ceramic. In detail, the substrate may be selected from the group consisting of silicon, glass, polystyrene, polymethylacrylate, polycarbonate, and ceramic.

The sample may be a biological sample derived from a subject, for example, a mammal including a human. In addition, the biological sample may be blood, whole blood, serum, plasma, lymph, urine, feces, tissue, cell, organ, bone marrow, saliva, sputum, cerebrospinal fluid, or a combination thereof. In detail, the biological sample may be urine or obtained from urine.

The biosensor may be capable of detecting a target material in a urine sample.

In an embodiment, in the case of the biosensor having the electrode surface modified with reduced graphene oxide nanosheet (rGON), the stability is significantly excellent in the urine environment as compared to the case where the electrode surface is not modified. In detail, it was confirmed that the shift of current signals is significantly reduced (see FIGS. 7A to 7B).

The electrode may be a working electrode, an insulating electrode, or a reference electrode. The electrode may be, for example, titanium nitride, silver, silver epoxy, palladium, copper, gold, platinum, silver/silver chloride, silver/silver ion, mercury/mercury oxide, or indium-tin oxide (ITO). Also, the reaction unit may include an insulating electrode formed on the substrate or the electrode. The insulating electrode may include an oxide film formed naturally or artificially. Examples of the oxide film are SnₓO_{y}, SiₓO_{y}, HₓfO_{y}, AlₓO_{y}, TaₓO_{y}, TiₓO_{y}, and the like (wherein x or y may be an integer from 1 to 5). The formation of the oxide film may be performed by a method known in the art. For example, the oxide film may be formed by depositing an oxide on the substrate by liquid phase deposition, evaporation, and sputtering.

On the insulating electrode, a test cell for accepting the sample may be attached. The test cell may be prepared from polydimethylsiloxane (PDMS), polyethersulfone (PES), poly(3,4-ethylenedioxythiophene), poly(styrenesulfonate), polyimide, polyurethane, polyester, perfluoropolyether (PFPE), polycarbonate, or a combination of these polymers.

The reaction unit may include a biological probe that specifically binds to the target material.

The term “biological probe” as used herein refers to a material capable of imparting functionalization to the reaction unit or a material specifically binding to the target material. The biological probe may include DNA, RNA, PNA, a nucleotide, a nucleoside, a protein, a polypeptide, a peptide, an amino acid, a carbohydrate, an enzyme, an antibody, an antigen, a receptor, a virus, a substrate, a ligand, a membrane, or a combination thereof.

The biological probe may be to detect one or more miRNA selected from the group consisting of miRNA21, miRNA1246, and let7b. In detail, the biological probe may include one or more biological probes to detect one or more miRNA selected from the group consisting of miRNA21, miRNA1246, and let7b. For example, the biological probe may be to detect miRNA in a urine sample, and in detail, to detect one or more selected from the group consisting of miRNA21 (SEQ ID NO: 4), miRNA1246 (SEQ ID NO: 5), and let7b (SEQ ID NO: 6), wherein the probe may be in the form of DNA, RNA, or PNA.

In an embodiment, as the biological probe to detect miRNA21, miRNA1246, and let7b, a PNA probe may be used. The PNA probe for detecting the miRNAs was named PNA-21, PNA-1246, and PNA-let7b, respectively, and a base sequence of the PNA probe for each miRNA may be represented by SEQ ID NO: 1 (PNA-21), SEQ ID NO: 2 (PNA-1246), and SEQ ID NO: 3 (PNA-let7b), respectively.

In an embodiment, the biosensor may be for detecting all of miRNA21, miRNA1246, and let7b. Here, the reaction unit of the biosensor may be divided into one or more regions, and a biological probe (for example, PNA-probe) capable of detecting one type of mlRNA may be separately attached.

In an embodiment, the biosensor may detect all of miRNA21, miRNA1246, and let7b with excellent accuracy. In particular, it was confirmed that the standard curve of the biosensor for miRNA21 and let7b showed better linearity, and that is, miRNA21 and let7b may be detected with better accuracy.

In addition, the biological probe may include a redox enzyme. The redox enzyme may refer to an enzyme that oxidizes or reduces a substrate, and for example, may include an oxidase, a peroxidase, a reductase, a catalase, or a dehydrogenase. Examples of the redox enzyme are blood glucose oxidase, lactase oxidase, cholesterol oxidase, glutamate oxidase, horseradish peroxidase (HRP), alcohol oxidase, glucose oxidase (GOx), glucose dehydrogenase (GDH), cholesterol estergenase, ascorbic acid oxidase, alcohol dehydrogenase, laccase, tyrosinase, galactose oxidase, bilirubin oxidase, and the like.

The reaction unit may include a linker for immobilizing the biological probe to the surface of the reaction unit or the surface of the electrode. The term “immobilization” as used here may refer that the biological probe forms a chemical or physical bond to a substrate or an electrode. The linker may include a compound including biotin, avidin, streptavidin, carbohydrate, poly L-lysine, a hydroxyl group, a thiol group, an amine group, an alcohol group, a carboxyl group, an amino group, a sulfur group, an aldehyde group, a carbonyl group, a succinimide group, a maleimide group, an epoxy group, and an isothiocyanate group, or a combination thereof. In detail, the linker may include pyrenebutyric acid N-hydroxy succinimide ester (PANHS).

The term “target material” as used herein is a target detection material that may exist in a sample, and may refer to a material that specifically binds to a biological probe. A detectable target material may include those that may be involved in a specific binding interaction with one or more biological probes that can participate in a sandwich, competition, or substitution assay configuration). Examples of the target material are an antigen such as a peptide (for example, a hormone), a haptene, a carbohydrate, a protein (for example, an enzyme), a drug, a microorganism, an antibody, and a hexane that can participate in a sequence-specific hybridization reaction with a complementary sequence, or a combination thereof. In detail, the target material may be miRNA.

The target material may be a prostate cancer marker, and in detail, may include one or more selected from the group consisting of miRNA21, miRNA1246, and let7b.

The term “prostate cancer (PCa)” as used herein refers to cancer occurring in the prostate. The prostate is a male reproductive organ about the size of a walnut located right below the bladder and in front of the rectum, and is responsible for producing and saving a part of semen. The prostate is adjacent to the bladder neck upward, i.e., an area from the bladder to the urethra, so that the prostate is fixed with the ligament puboprostaticum frontward and is also fixed with the urogenital diaphragm downward. Most of cancers occurring in the prostate are adenocarcinoma (cancer of glandular cells) that occurs in prostate cells, and cancer types can be classified according to the degree of differentiation of a tumor tissue and cell characteristics.

The term “biomarker” or “marker” as used herein generally refers to an indicator that can detect changes in the body by using an organic biomolecule, such as a protein, a nucleic acid (e.g., DNA, mRNA, miRNA, and the like), a metabolite (e.g., lipid, glycolipid, glycoprotein, sugar, and the like). In detail, in the case of a specific disease or cancer, the term refers to an indicator that can distinguish a normal state or a pathological state or predict a treatment response, and that can be objectively measured. Depending on usage, the biomarker may include a target marker that identifies the presence of a drug target, a diagnostic marker for diagnosing the presence or absence of disease, a predictive marker that can distinguish a responder group and a non-responder group with respect to a specific drug, a surrogate marker that can monitor a drug therapeutic effect, a prognostic biomarker that inform the prognosis of disease, and the like.

The biosensor may be for detecting a prostate cancer marker, and in detail, may be for diagnosis of prostate cancer.

The biosensor may quantitively detect a target material in a sample. In detail, a detection signal appearing in the biosensor is generated in a concentration-dependent manner of the target material in the sample, and thus based on a concentration-detection signal reference graph, the target material in the sample may be quantitatively analyzed.

In an embodiment, the biosensor may quantitively detect a prostate cancer marker, in detail, one or more miRNAs selected from the group consisting of miRNA21, miRNA1246, and let7b. In detail, the detection signal for miRNA is proportional to the concentration of miRNA in the sample, and in this regard, the concentration of miRNA in the sample may be quantitatively analyzed through the concentration-detection signal reference graph.

In an embodiment, the biosensor may diagnose whether or not prostate cancer develops or is at risk of development by detecting miRNA21, miRNA1246, and/or let7b. In particular, it was confirmed that let7b shows a remarkably superior difference in voltage fluctuations between a patient with prostate cancer and a normal person to other types of miRNA, and thus let7b may exhibit better effects of diagnosis of prostate cancer. Therefore, the biosensor may be used for diagnosis of prostate cancer.

The biosensor may further include a signal converter (signal processing unit) that recognizes the interaction and converts it into an electrical signal. Here, the reaction unit of the biosensor may be separable from the signal converter.

The signal converter may include a field-effect transistor (FET), and may be formed by a connection between the electrode of the reaction unit and an upper gate electrode of the FET. Such a connection may be, for example, in the form of a plug.

In the reaction unit, a sample may be introduced through the electrode, the biological probe, and the test cell for accepting target material, and a target material present in the sample binds to the biological probe to generate a chemical potential gradient in the test cell. The term “chemical potential gradient” as used herein may refer to a concentration gradient of an active species. When such a gradient exists between two electrodes, a potential difference may be detectable when a circuit is open. Meanwhile, when the circuit is closed, the current may flow until the gradient disappears. The chemical potential gradient may refer to any potential gradient resulting from the application of the potential difference or current flow between the two electrodes.

The FET may include: a substrate; an insulating layer; a source electrode and a drain electrode that are spaced apart from each other; a gate electrode; and a channel layer arranged between the source electrode and the drain electrode. In an embodiment, the FET may be a dual-gate FET including: a bottom gate electrode; a bottom insulating film formed on the bottom gate electrode; a source and a drain that are formed on the bottom insulating film and spaced apart from each other; a channel layer formed on the bottom insulating film and arranged between the source and the drain; a top insulating film formed over the source, the drain, and the channel layer; and a top gate electrode formed on the top insulating film.

A small surface potential voltage difference generated in the reaction unit may greatly amplify a threshold voltage change of the lower FET due to ultra-electrostatic coupling occurring in a dual-gate ion-sensitive field-effect transistor (ISFET) including a channel layer. Here, an amplification factor may be determined by a thickness of the bottom insulating film, a thickness of the channel layer, and a thickness of the insulating film of the top gate. As the thickness of the bottom insulating film increases and the thicknesses of the top insulating film and the channel layer decrease, the size of the amplification factor may increase.

The channel layer may be an ultra-thin layer, and for example, may have a thickness of 10 nm or less, 9 nm or less, 8 nm or less, 7 nm or less, 6 nm or less, 5 nm or less, or 4 nm or less.

Within the thickness range of the channel layer, due to the strong electric field of the bottom gate electrode induced in a ultra-thin body, ultra-electrostatic coupling that can be controlled under all conditions may occur up to the top interface. Accordingly, electrons and holes induced in the top gate interface may be also controlled, and leakage current may be blocked. In addition, by allowing a stable amplification factor, linear response upon the surface potential, hysteresis, and drift phenomena may be improved, and the electrostatic coupling of the top and bottom gates may be sustained. In addition, within the thickness range of the channel layer, the transistor including the ultra-thin channel layer may enhance ion sensing power by allowing use of an amplification factor that is larger than the existing transistor. In addition, within the thickness range of the channel layer, the transistor including the ultra-thin channel layer may also improve stability compared to a conventional transistor. The variable amplification factor seen in the thick channel layer may combine with a leakage current element induced in the top interface, resulting in deterioration of a device due to ion damage. Meanwhile, by allowing a constant amplification factor, the transistor according to an embodiment in which the leakage current is controlled may minimize the ion damage. In addition, when a bottom insulating film is excessively thick in the existing transistor, a phenomenon that the bottom electric field cannot control all of channel regions occurs, and accordingly, the electrostatic coupling of the top and bottom gates is weakened. However, the transistor including the ultra-thin channel layer according to an embodiment may obtain a large amplification factor while maintaining the electrostatic coupling. The electrostatic coupling phenomenon of the top and bottom gates occurs when the top channel interface is completed depleted. Here, in the existing transistor, amplification does not occur because the electric field of the bottom gate does not control a top channel.

The channel layer may include any one selected from the group consisting of an oxide semiconductor, an organic material semiconductor, polycrystalline silicon, and single crystal silicon. When the channel layer includes any one selected from the group consisting of a semiconductor, an organic material semiconductor, polycrystalline silicon, and single crystal silicon, electrostatic coupling between the top and bottom gates occurs, and a high-sensitive sensor may be manufactured. Accordingly, a transparent and flexible sensor may be provided. The channel layer is not limited in width and length, and in the dual-gate structure described above, the electrostatic coupling phenomenon may be utilized by using the top and bottom gate electrodes.

In addition, in the sensor, an equivalent oxide thickness of the top insulating film may be smaller than that of the bottom insulating film. For example, a thickness of the top insulating film may be about 25 nm or less, and a thickness of the bottom insulating film may be about 50 nm or more. When the equivalent oxide thickness of the top insulating film is smaller than that of the bottom, signal sensitivity amplification may be caused.

The top insulating film and the bottom insulating film may each include an oxide film formed naturally or artificially. Examples of the oxide film are SiₓO_{y}, HₓfO_{y}, AlₓO_{y}, TaₓO_{y}, TiₓO_{y}, and the like (wherein x or y may be an integer from 1 to 5). The oxide film may have a single-, double-, or triple-stacked structure. That is, by increasing a physical thickness and decreasing the equivalent oxide thickness of the top insulating film, the sensitivity of the sensor may be amplified while the deterioration caused by leakage current may be prevented.

The dual-gate ISFET according to an embodiment may have a structure including a top-field transistor including a top insulating film and a bottom-field transistor including a bottom insulating film at the same time in a single device. Depending on each operation mode, the top and bottom gates may operate independently. When the top and bottom gates of the device are used at the same time, an electrostatic coupling phenomenon may be observed due to the structurally distinct characteristics of the dual-gate structure, thereby establishing correlation between the top and bottom field transistors. In such a dual-operation mode, the bottom gate may be used as a main gate. Therefore, the transistor according to an embodiment may operate in a dual-gate mode.

The sensor may include: a plurality of reaction units for detecting a plurality of target materials; and a plurality of transistors. The sensor may include: a plurality of the reaction units; and a plurality of the ISFETs, wherein each of the plurality of the reaction units may be electrically connected to each of the plurality of the ISFETs. In the plurality of the transistors, a plurality of sources may be commonly grounded, a plurality of top gate electrodes may be commonly grounded, and a common voltage may be applied to a plurality of bottom gate electrodes. For example, sources of the first transistor and the second transistor and reference electrodes of the first reaction unit and the second reaction unit may be commonly grounded. For example, a constant common voltage may be applied to the bottom electrodes of the first transistor and the second transistor. In addition, a plurality of drains in the plurality of the transistors may have a parallel structure. For example, drains of the first transistor and the second transistor may have a parallel structure. In addition, the plurality of the reaction units may be independently immobilized with different biological probes. The plurality of the transistors may detect the same or different target material signals from the plurality of the reaction units, amplify the signals, and output the signals by using a semiconductor parameter analyzer.

The signal converter may be electrically connected to the transistor, and may further include an arithmetic module for determining an amount of the target material in the sample based on the potential difference measured from the transistor. The arithmetic module may be for determination of the target material. The term “determination of a target material” as used herein may refer to a qualitative, semi-quantitative, and quantitative process for evaluating a sample. In a qualitative evaluation, results indicate whether a target material is detected in a sample. In a semi-quantitative evaluation, results indicate whether a target material is present above some predefined threshold values. In a quantitative evaluation, results are numerical representations of a target material present. In addition, the conversion of the measured value may use a look-up table that converts a specific value of current or potential into a value of a target material depending on a specific device structure and a correction value for the target material. The arithmetic module may be determined by measuring a potential difference according to a known concentration of the target material. For example, the arithmetic module may be to determine the amount of the target material in the sample with respect to a normal control group.

The biosensor may further include a display unit for displaying a result. The display unit may further include a frame having a display for displaying results and one or more control interfaces (for example, a power button or a scroll wheel). The frame may include a slot for receiving a sensor. Inside the frame, a circuit may be included to apply a potential or a current to the electrode of the sensor when the sample is provided. A suitable circuit that may be used in the measuring meter may be, for example, an ideal voltage meter capable of measuring the potential across the electrode. A switch that opens when the potential is measured or closes for measurement of the current may be also provided. The switch may be a mechanical switch (for example, a relay) or a solid-state switch. The circuit may be then used to measure a potential difference or a current difference. As will be understood by one of ordinary skill in the art, other circuits including simpler but more complex circuits may be used to achieve the application of a potential difference or a current difference or both.

The biosensor may be equipped with a communication means, and thus may be configured to enable transmission and receiving information with an external server or terminal unit. The communication means may employ a wired or wireless communication means. In this regard, wired communication using a cable connection means may be used, and wireless communication means including, not only a Bluetooth module or a Zigbee module, but also 5G, 4G, LTE, UWB, WiFi, WCDMA, USN, and IrDA modules may be used.

The terminal unit may include a communicator such as a computer, a laptop, a smart phone, a PDA, a measuring instrument or a control device having a separate communication function. The terminal unit may be equipped with a central processing apparatus, and may be based on an operating system (OS) that can run a software such a computer program, and application program, and the like. Therefore, the terminal unit may loaded with an application program that can interpret, analyze, and process measurement data of an analyte (target material) in a sample provided from the sensor, thereby performing a function of interpreting, analyzing, and processing the measurement data of the analyte in the sample. In addition, the terminal unit may perform a function of displaying the measurement data of the analyte in the sample, or displaying data after interpreting, analyzing, and processing the measurement data of the analyte in the sample. In addition, the terminal unit is connected or interlocked with a control unit of the sensor, and thus may be perform a function of operating and controlling the sensor.

Another aspect provides a method of detecting a target material, the method including: contacting a sample with the biosensor; and observing an electrical conductivity change in the biosensor. The same contents as described above also apply to the descriptions of the method.

The term “detection” as used herein may refer to discovery or confirmation of the appearance or existence of a target material, and for example, may refer to identification of a target material or quantification of a target material in a sample.

The detection method may be an electrochemical method of measuring a current or a potential generated through an interaction between a target material and a biological probe.

Another aspect provides a method of providing information on diagnosis of prostate cancer, the method including measuring an expression level of a prostate cancer marker by using the biosensor, from a biological sample isolated from a subject. The same contents as described above also apply to the descriptions of the method.

The term “diagnosis” as used herein refers to identification of the presence or properties of pathological states. Regarding the purpose of the present disclosure, the diagnosis may refer to determination of the development of prostate cancer.

The term “prognosis” as used herein refers to prediction of disease progression and recovery, and also refers to a prospect or a preliminary evaluation. Regarding the purpose of the present disclosure, the diagnosis may refer to determination of success of treatment, survival, recurrence, metastasis, drug response, resistance, and the like, in a corresponding subject after treatment of prostate cancer. That is, the prognosis means the prediction of medical consequences (for example, organ viability, disease-free survival rates, and the like), and may include positive prognosis or negative prognosis. The negative prognosis includes disease progression or mortality in terms of recurrence, tumor growth, metastasis, drug resistance, or the like, and the positive prognosis includes remission of disease in a disease-free state or the like, or improvement or stabilization of a disease such as tumor regression.

The term “prediction” as used herein refers to assumption in advance for medical consequences. Regarding the purpose of the present disclosure, the prediction may mean assuming in advance the progress of a disease (for example, disease progression, improvement, cancer recurrence, tumor growth, drug resistance, or the like) of a patient diagnosed with prostate cancer.

The term “subject” as used herein refers to any organism that has developed or is likely to develop prostate cancer, and specific examples thereof are mammals including dogs, cats, mice, rats, monkeys, cattle, pigs, mini-pigs, livestock, humans, and the like, farmed fish, and the like. However, the subject is not limited thereto.

The prostate cancer marker may include one or more selected from the group consisting of miRNA21 (SEQ ID NO: 4), miRNA1246 (SEQ ID NO: 5), and let7b (SEQ ID NO: 6).

The measuring of the expression level of the prostate cancer marker by using the biosensor may include: contacting the sample with a biosensor capable of detecting a prostate cancer marker; and observing a change in electrical conductivity of the biosensor. Here, the biosensor the one disclosed herein including a biological probe capable of detecting the prostate cancer marker.

The method may further include: measuring an expression level of a prostate cancer marker in a biological sample isolated from a control group; and comparing the expression levels of the subject and the control group.

The term “control group” as used herein may refer to a general subject who has not developed prostate cancer, a non-prostate cancer patient group, a non-patient group, or the like.

The method may further include determining whether or not the subject develops prostate cancer or predicting a high risk of developing prostate cancer, when the expression level of the prostate cancer marker in the subject is higher than that of the control group.

### Advantageous Effects

A biosensor according to an aspect can effectively detect a target material in a urine sample, and has an advantage in that use of the biosensor enables diagnosis of prostate cancer in a non-invasive, fast, accurate manner.

### DESCRIPTION OF DRAWINGS

FIG. 1A is a schematic diagram showing a urine miRNA sensing system in urine of a patient based on a disposable and switchable electrical sensor, and FIG. 1B is a diagram showing a process of chemical preparation and surface modification of a disposable sensor chip.

FIG. 2 is a diagram illustrating a reaction unit of a biosensor according to an embodiment.

FIG. 3A is a diagram showing a morphological image and a line-profile result for a thickness observed with an atomic force microscope, wherein a width and a height of rGON were measured to be about 500 nm and about 1.5 nm, respectively, and FIG. 3B is a diagram showing a result from UV-vis-NIR absorbance spectrum, showing red shift of the absorbance by π-π transition of the aromatic carbon bonds in rGON by partial restoration of the sp2 carbon structural domain.

FIG. 4 is a diagram showing the Raman spectrum results of ITO, ITO/SnO₂, and ITO/SnO₂/rGON, wherein the Raman spectrum of ITO/SnO₂/rGON shows that D band and G band which are typical characteristics of rGON are located at 1360 cm⁻¹ and 1600 cm⁻¹, respectively.

FIG. 5 is a diagram showing characteristic analysis results according to surface modification of a disposable sensor chip, and provides atomic force microscope phase images of SnO₂ (FIG. 5A), rGON (FIG. 5B), PANHS (FIG. 5C), a PNA probe (FIG. 5D), and a line profile result (FIG. 5E). As a result of the line-profile, it is confirmed that heights of SnO₂, SnO₂/rGON, SnO₂/rGON/PANHS, and SnO₂/rGON/PANHS/PNA gradually increase from 0 nm to 3.5 nm. FIG. 5F is a diagram showing the relative average surface potentials of SnO₂, SnO₂/rGON, SnO₂/rGON/PANHS, and SnO₂/rGON/PANHS/PNA, wherein, by using a neutral representative of HOPG, 54.4±0.5 mV, −146.7±0.7 mV, −116.6±0.3 mV, and −119.5±0.4 mV are measured respectively.

FIG. 6A is a diagram showing transfer curve of an miRNA sensor measured at a bottom gate sweep, and FIG. 6B is a diagram showing pH sensitivity of an miRNA sensor in a buffered solution having pH 3 to pH 11.

FIG. 7A is a diagram showing drift characteristics of an miRNA sensor measured in human urine and PBS for 60 minutes, and FIG. 7B is a diagram showing variation of V_{BG} in human urine and PBS after washing for 20 minutes.

FIG. 8 is a diagram showing a stability test result of a disposable sensor chip.

FIG. 9 is a diagram showing changes in the electrical signals for target miRNA and miRNA having a sequence in which one base is mismatched, in a disposable sensor chip equipped with a probe capable of detecting miRNA.

FIG. 10 is a diagram showing a difference in electrical signals among wells of a disposable sensor chip, wherein, before use, as a result of I_{D}-V_{G} analysis for each independent well immobilized with a probe targeting miRNA, no difference in the electrical signals among the wells is confirmed.

FIG. 11 is a diagram showing I_{D}-V_{G} curve results of the miRNA sensor according to concentrations of three target miRNAs, i.e., miR21 (FIGS. A and D), miR1246 (FIGS. B and E), and Let7b (FIGS. C and F), wherein FIGS. 11A, 11B, and 11C show the results measured in 1× PBS, and FIGS. 11D, 11E, and 11F show the results measured in human urine samples.

FIG. 12 is a diagram showing a standard curve of the miRNA sensor for three miRNAs, i.e., miR21 (FIGS. A and D), miR1246 (FIGS. B and E), and Let7b (FIGS. C and F), at a dynamic dose in a range of 10 fM to 10 nM, wherein FIGS. 12A, 12B, and 12C show the results measured in 1× PBS, and FIGS. 12D, 12E, and 12F show the results measured in human urine samples.

FIG. 13A is a diagram illustrating a disposable sensor chip for real-time monitoring of three different miRNAs per patient at the same time, and FIG. 13B is a diagram showing a voltage difference between a patient with prostate cancer and a patient without prostate cancer. Higher levels of the cancer-specific miRNA were monitored in a cancer patient group than in a normal group, and such monitoring results are consistent with those from the pathological diagnosis.

FIG. 14 is a diagram showing results of confirming miRNA expression levels by real-time qPCR, wherein relative expression levels of miRNAs (i.e., miR21, miR1246, and Let7b) are shown between urine of a cancer patient (D) and urine of a normal person (I), (error bars represent mean±SE (n=3) , ****p<0.0001, ***p<0.001).

### MODE FOR INVENTION

Hereinafter, the present disclosure will be described in more detail with reference to Examples below. However, these Examples are for illustrative purposes only, and the scope of the present disclosure is not intended to be limited by these Examples.

### Example 1: Information of Urine Sample of Patient

All the urine samples and clinical results were obtained from patients after informed consent according to the guidelines of Korea University College of Medicine. The protocol for human urine analysis was approved by the ethics committee of Korea University College of Medicine and Korea University Anam Hospital (IRB no. 2018AN0332), and the human urine analysis was performed according to the approved guidelines. Urine samples used herein were as follows:

Non-cancer urine (77-, 62-, 62-, and 78-years-old), prostate cancer urine (69-years-old, GS (3+3); 80-years-old, GS (3+3); 58-years-old, GS (4+4); 80-years-old, GS (4+4); 56-years-old, GS (4+5); 69-years-old, GS (4+4)).

### Example 2: Preparation of Urine miRNA Sensing System (Biosensor)

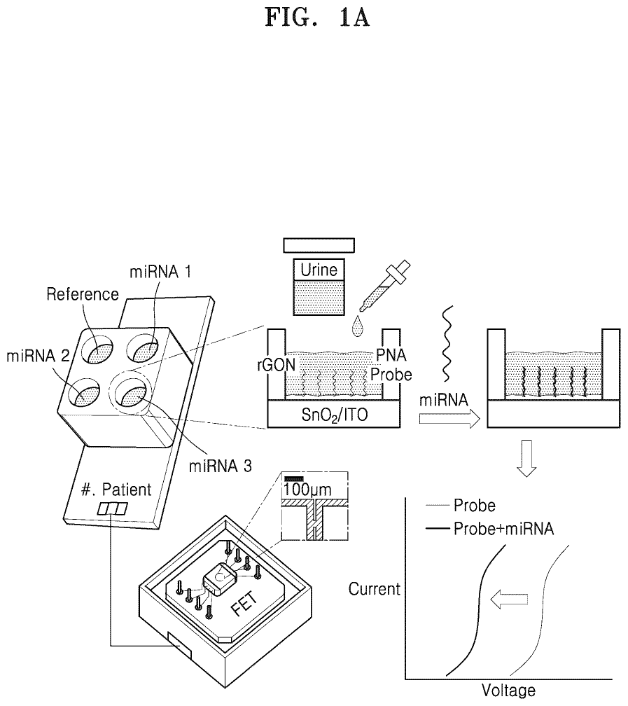

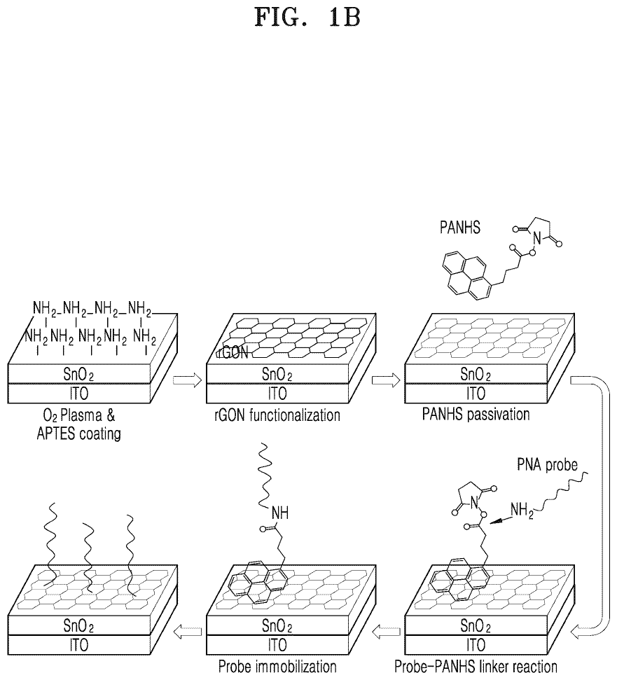

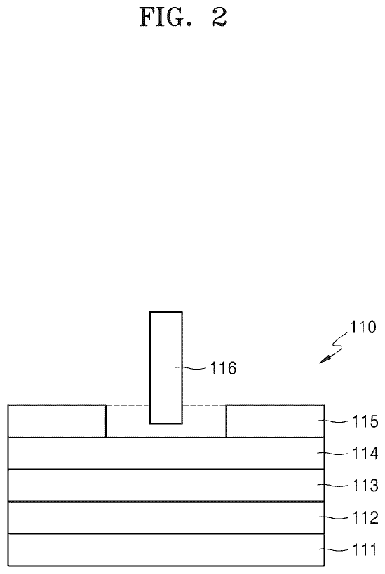

In the present disclosure, a label-free urine miRNA sensing system and a non-invasive clinical diagnostic method using the same were provided (see FIG. 1A), wherein the system was based on a disposable and switchable graphene-based electrical sensor having high sensitivity and high specificity in urine of a patient. A sensing module of the sensor was easily connected to the body of a field-effect transistor (FET), and was able to rapidly and accurately detect an miRNA marker in a urine sample obtained from a patient with prostate cancer. To configure the miRNA sensing system, a surface of a disposable sensor chip functionalized with reduced graphene oxide nanosheet (rGON) was designed, and then, peptide nucleic acid (PNA) was immobilized on the surface (see FIG. 1B).

Meanwhile, the FET corresponding to a signal processing unit (signal converter) of the biosensor was prepared according to the method described in a known document (Adv. Healthcare Mater. 2017, 6, 1700371).

In addition, to prepare a reaction unit (sensing unit) of the urine miRNA sensing system of the present disclosure, the following procedure was performed.

### 2-1: Preparation of ITO/SnO₂Substrate

To prepare a disposable sensor chip corresponding to the reaction unit (sensing unit) of the biosensor, a glass piece having a size of about 19 mm×27 mm×0.7 t mm (width×length×thickness) was used as a substrate. After performing a standard RCA cleaning process, an indium tin oxide (ITO) film (300 nm) as a working electrode for measuring an electrical potential difference was deposited on a surface of the substrate by using an E-beam evaporator. Next, as an insulating electrode, an SnO₂ oxide film was deposited on the ITO film to a thickness of about 45 nm by using an RF sputter. Here, the RF power was about 50 W. Afterwards, a sputtering process was performed thereon under an Ar gas condition having a flow rate of about 20 sccm and a pressure condition of about 3 mtorr. Next, to fabricate a polydimethylsiloxane (PDMS) well, a base and a curing agent (Sygard 184) were formulated at a ratio of 10:1, and this mixture was cured at 60° C. for 3 hours. The PDMS well was treated with O₂ plasma (30 sccm of O₂ gas flow at 70 W for 1 minute, Plasma System Cute, Femto Science), and then, attached to the insulating electrode, so as to fabricate a reaction unit. For a test cell, it was fabricated as an extended gate (EG) with four wells. Furthermore, as a reference electrode, a silver/silver chloride electrode was used.

### 2-2: Preparation of Reduced Graphene Oxide Nanosheet (rGON)

An rGON for use in the disposable sensor chip of the present disclosure was prepared as follows.

First, graphene oxide (GO) was synthesized from graphite according to the modified Hummers method, and was additionally sonicated at 4° C. for 6 hours.

Next, under thermal conditions, an rGON was prepared by using L-ascorbic acid. In detail, 50 mg of L-ascorbic acid was dissolved in of 50 mL (0.1 mg/mL) of an aqueous GO solution, and the mixed solution was vigorously stirred at 120° C. for 24 hours. Then, the resulting solution was cooled at room temperature for 80 hours while maintaining stirring, and an rGON was collected by dialysis using a 10 kDa membrane for 48 hours. For the next experiment, the resulting solution was resuspended in deionized autoclaved water (DW).

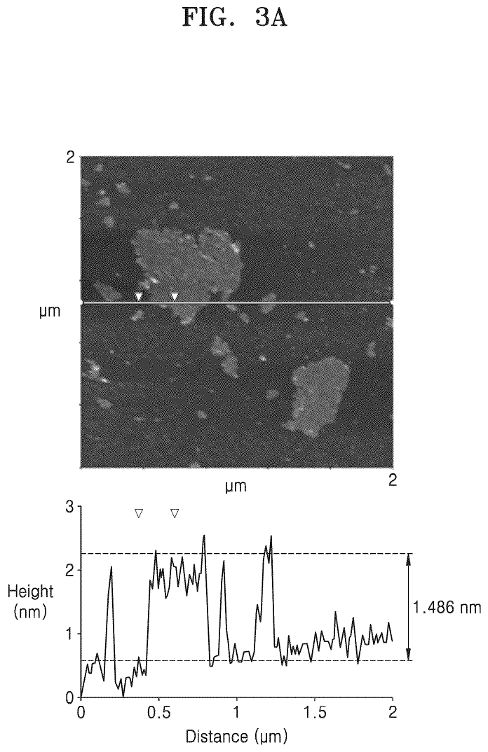

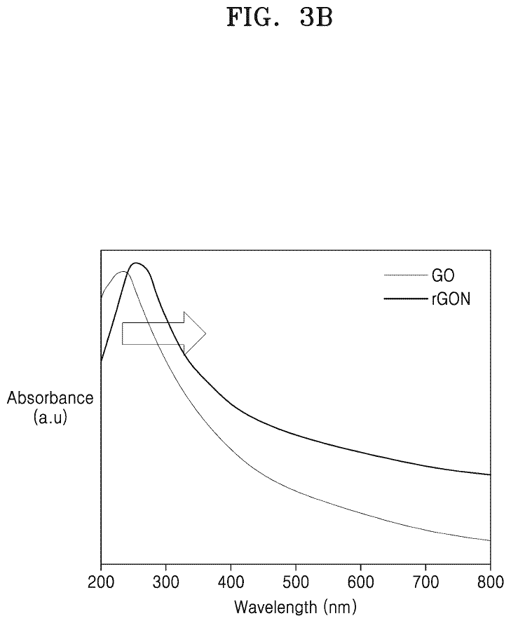

As a result of observing the size and phase of the prepared rGON with an atomic force microscope (AFM) (XE-100, Park System), a line-profile image thus obtained showed that a diameter and a thickness of the rGON were about 500 nm and about 1.5 nm, respectively. Accordingly, it was confirmed that a typical rGON monolayer was formed (see FIG. 3A). Next, as a result of analyzing absorbance spectrum of ultraviolet-visible-near-infrared (UV-vis-NIR) of GO and rGON by using a UV-2550 spectrophotometer (Shimadzu, Japan), typical absorbance peaks of GO were observed at 235 cm⁻¹ by π-π transition of the aromatic carbon bonds, the rGON showed a red shift, and the broad light absorbance increased in the visible and near-infrared ray regions. Accordingly, it was confirmed that partial restoration of the sp2 carbon structural domain was supported (see FIG. 3B).

Therefore, based on the results above, it was confirmed that the rGON for use in the disposable sensor chip was successfully prepared.

### 2-3: Modification of ITO/SnO₂ Substrate Using rGON

To functionalize the SnO₂ surface of the SnO₂/ITO substrate with rGON, the following experiment was performed.

In detail, to form a hydrophilic hydroxyl group (—OH) on the SnO₂ substrate, the surface of the disposable sensor chip was treated with an O₂ plasma system at 70 W for 1 minute. Afterwards, to form an amine group (—NH₂) which is a positively charged functional group, an ethanol solution containing 5% 3-aminopropyltriethoxysilane (APTES) was treated thereon for 1 hour. The disposable sensor chip was then washed with ethanol, and backed at 120° C. for 30 minutes. Next, by immersing with rGON (0.1 mg/mL) at room temperature, the surface of the disposable sensor chip was functionalized with rGON by electrostatic interactions between a positive charge of the SnO₂ surface and a negative charge of the rGON. The rGON was deposited as a two-dimensional thin film by using a drop-casting method which is a simple and rapid deposition method performed at room temperature.

The rGON may be easily functionalized with a peptide nucleic acid (PNA) probe to capture miRNA over a large surface area by providing a restored sp2 carbon structural domain. In addition, the rGON improved the conductivity and electron mobility, thereby lowering intrinsic electrical noise and enabling signal improvement without a cofactor. Therefore, the disposable sensor chip on which the rGON was deposited was more sensitive to electrical signals for capturing miRNA included in urine.

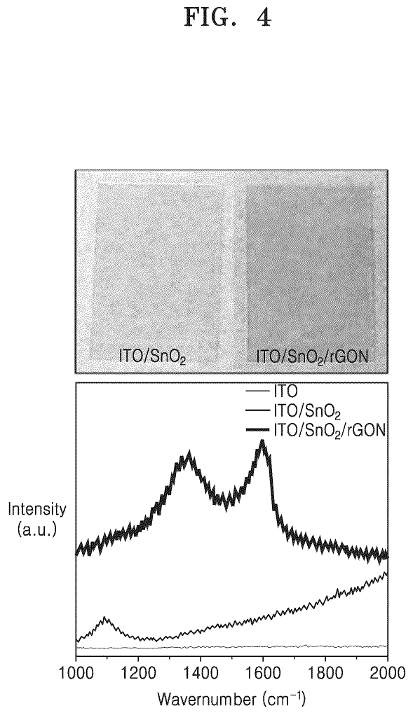

To analyze characteristics of the prepared ITO/SnO₂/rGON, the Raman spectrum of the ITO/SnO₂/rGON was analyzed using LabRAM HR UV-vis-NIR(Horiba Jobin Yvon, France) using 20 mW Arion CW laser (514.5 nm) as an excitation source focused via a BXFM confocal microscope equipped with an object lens (50×, numerical aperture=0.50). As a result, it was confirmed that two main bands, D and G bands, located at 1360 cm⁻¹ and 1600 cm⁻¹, respectively, which are typical characteristics of rGON appeared in the Raman spectrum of the ITO/SnO₂/rGON (see FIG. 4), thereby confirming the functionalization of the substrate with the rGON.

### 2-4: Probe immobilization

To immobilize a probe on the ITO/SnO₂/rGON substrate prepared in Example 2-3, the following experiment was performed.

First, N,N-dimethylmethanamide (DMF) containing 1 μM of pyrenebutyric acid N-hydroxy succinimide ester (PANHS) was added to the substrate and incubated at room temperature for 6 hours, thereby stabilizing/passivating the PANHS by π-π interactions on the surface of the rGON. Subsequently, a washing process was performed on the substrate twice by using DMF and 1×PBS. 1×PBS containing 1 μM PNA probe capable of binding to target miRNA was cultured overnight at room temperature, followed by a washing process twice by using distilled water and the 1×PBS. At the terminus of the PNA probe, neutrally charged polyethylene glycol (PEG)-amine and additional carbon chains are present. In this regard, the PNA may be actively immobilized on the surface of the rGON by using the PANHS as a linker, and furthermore, aggregation which is important when using the PNA probe may be prevented. In addition, the PNA probe having a neutral peptide backbone provides greater sequence-specific affinity and stability for miRNA, compared to a commonly used DNA-based probe. After the PNA probe was hybridized with a target miRNA to form PNA-miRNA, the shift of electrical signals in a device was determined by the electron charge of miRNA. The sequences of the PNA probe and target miRNA used in the present disclosure are shown in Table 1.

TABLE 1

| | | SEQ ID |
|---|---|---|
| | | NO. |
| PNA probe | Sequence (N → C) | |
| PNA-21 | TCA ACA TCA GTC TGA TAA GCT A | 1 |
| PNA-1246 | CCT GCT CCA AAA ATC CAT T | 2 |
| PNA-let7b | AAC CAC ACA ACC TAC TAC CTC A | 3 |
| Target | | |
| miRNA | Sequence (5′ → 3′) | |
| miRNA21 | UAG CUU AUC AGA CUG AUG UUG A | 4 |
| miRNA1246 | AAU GGA UUU UUG GAG CAG G | 5 |
| let7b | UGA GGU AGU AGG UUG UGU GGU U | 6 |
| mismatch | UAG CUU AUC AUA CUG AUG UUG A | 7 |

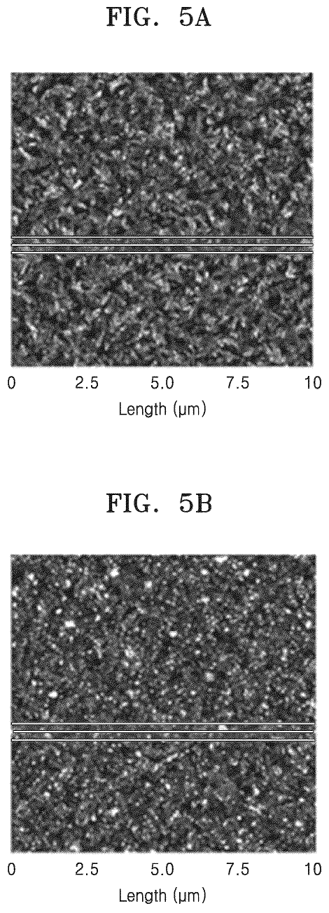

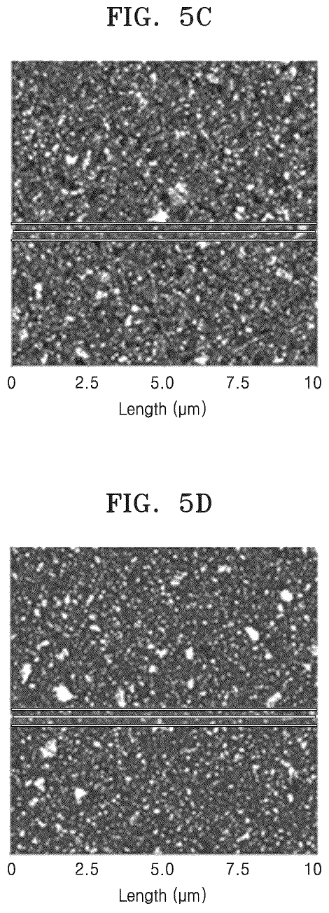

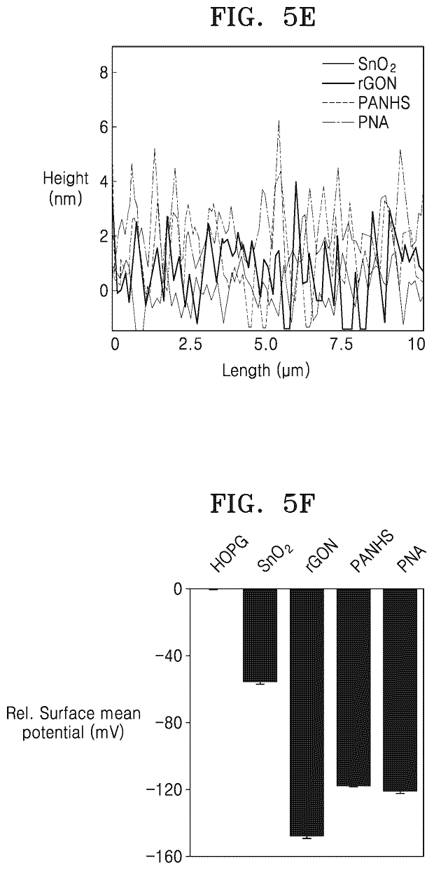

To analyze characteristics of the prepared SnO₂/rGON/PANHS/PNA substrate, AFM line scan analysis was performed. Consequently, it was confirmed that the SnO₂, SnO₂/rGON, SnO₂/rGON/PANHS, and SnO₂/rGON/PANHS/PNA each had a height gradually increasing from 0 to 3.5 nm (see FIGS. 5A to 5E). In addition, the surface average potentials of SnO₂, SnO₂/rGON, SnO₂/rGON/PANHS, and SnO₂/rGON/PANHS/PNA were measured by using the zetasizer ZSP (Malvern, UK). Consequently, by using a neutral representative of highly oriented pyrolytic graphite (HOPG), the surface average potentials were determined to be −54.4±0.5 mV, −146.7±0.7 mV, −116.6±0.3 mV, and −119.5±0.4 mV, respectively (see FIG. 5F).

Summarizing the results above, it was confirmed that the results above were consistent with the results of the stepwise surface functionalization using the rGON and PNA of the disposable sensor chip of the miRNA sensing platform of the present disclosure.

### Example 3: Evaluation of Electrical Performance and Stability of miRNA Sensor

### 3-1: Evaluation of Sensitivity of miRNA Sensor

Since the miRNA sensing system of the present disclosure identifies the presence of a target biomarker by measuring a voltage difference between a reference and a sample at a constant and stable current, the current stability from a source to the drain of the device of the present disclosure is considered important. Thus, by investigating the electrical characteristics of the sensor in the buffered solution and using a bottom gate sweep in the modified disposable sensor chip of the miRNA sensor, a transfer curve (I_{D}-V_{G}) and pH sensitivity were determined.

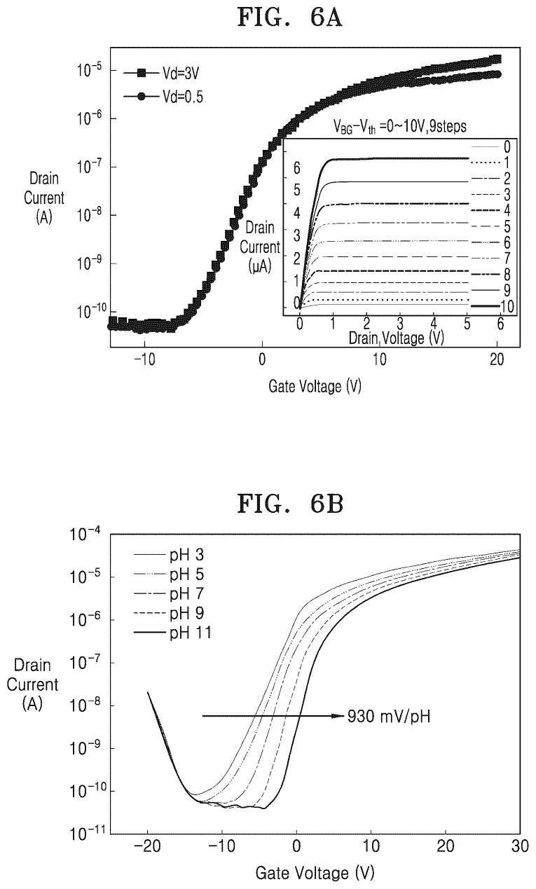

In detail, FIG. 6A shows a I_{D}-V_{G} curve and an output characteristics curve (I_{D}-V_{D}) of the sensor by the gate sweep (−13 V to 20 V) in the 1×PBS. During dual-gate operation, the sensor exhibited a low threshold voltage (Vₜₕ), an on/off current ratio (>10⁵), and subthreshold swing (1746 mV/dec). In addition, in FIG. 6B, highly improved pH sensitivity at an altitude of 930 mV/pH, which is 15 times greater than the Nernst limit (59 mV/pH), was observed at various pH values (between pH 3 and pH 11). Based on these results, it was confirmed that the miRNA sensor to which the modified disposable sensor chip was applied had high sensitivity, thereby being available as a biosensor.

### 3-2: Evaluation of Durability and Stability of miRNA Sensor

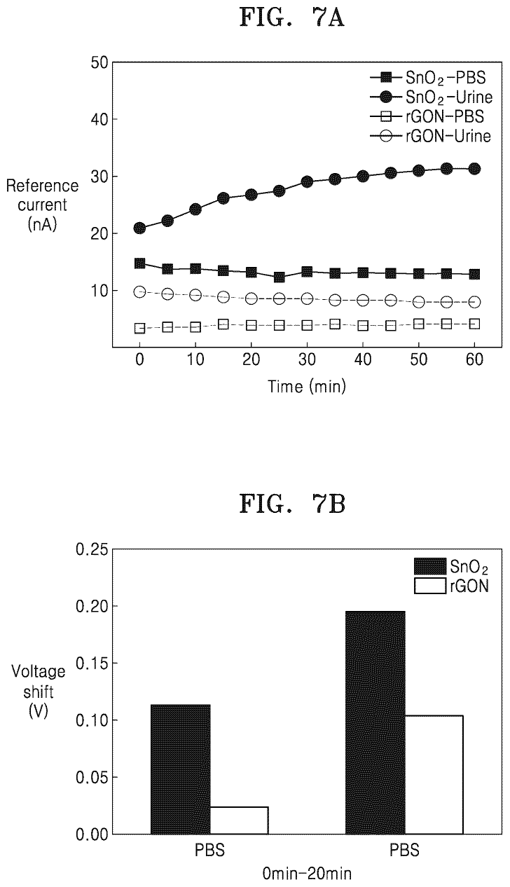

Device durability and stability are essentially considered in the urine environments, the real-time drift characteristics as a function of the current signal were evaluated in human urine and 1×PBS (see FIG. 7). In detail, compared to an unmodified disposable sensor chip, the sensor including the disposable sensor chip modified with rGON showed an extremely small shift in the current signal. In both 1×PBS and undiluted urine, a low change in the voltage shift was shown, whereas the response was stable. In addition, stable results were obtained even when electrical signal measurements were performed in urine or 1×PBS for 20 minutes. In this regard, it was confirmed that, unlike the unmodified SnO₂ substrate of the disposable sensor chip, the surface modification with rGON was able to significantly suppress interference by various pH conditions and undesirable factors such as ascorbic acid and urea compounds which make detection of urine miRNA difficult. These results supported import stability as a stable and sensitive real-time biosensor.

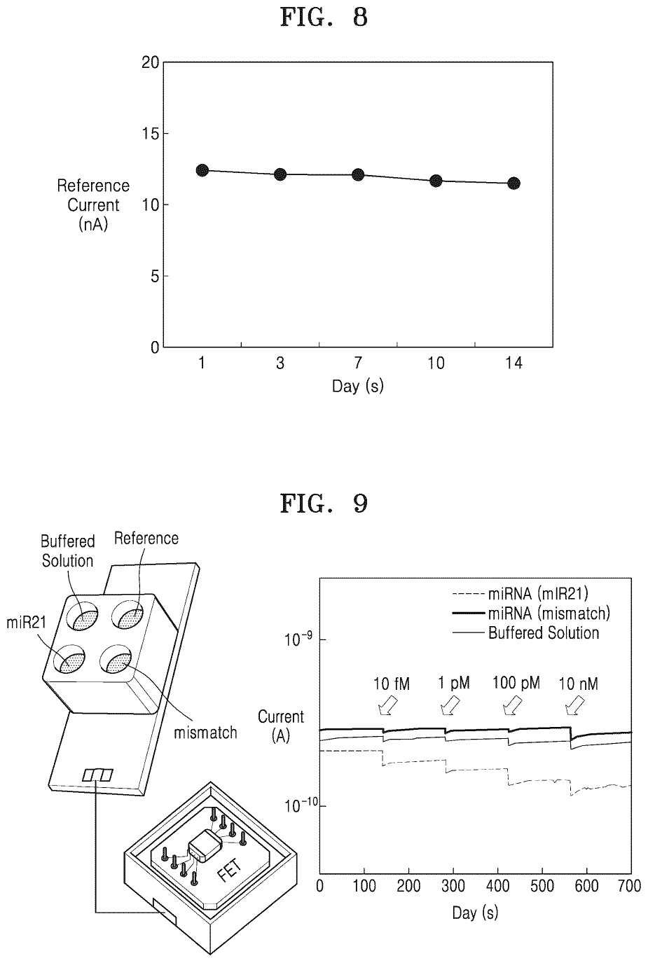

In addition, it was confirmed that, even when the prepared disposable sensor chip was stored at 4° C. for 2 weeks, 95% or more of the original reaction was still maintained (see FIG. 8), thereby exhibiting excellent durability and stability of the sensor chip.

### 3-3: Sensitive Evaluation According to miRNA Concentration

By monitoring the drain current with a time function, the sensitivity of the sensor to various concentrations (0 nM to 10 nM) of miRNA to be detected was confirmed. Here, a PNA-21 probe (miR21) was applied to the disposable sensor chip to detect miRNA21. As a result, throughout the measurement process, no significant change in current signal was detected in miRNA having a sequence in which only one base was mismatched and in the buffered solution itself. However, it was confirmed that the target miRNA to be detected was detected by reacting in a wide concentration range (see FIG. 9). Based on these results, it was confirmed that the miRNA sensor of the present disclosure had excellent specificity to the target miRNA by a sequence-specific method.

### Example 4: Evaluation of miRNA Detectivity of miRNA Sensor

To evaluate the sensor performance such as miRNA detectivity of the miRNA sensing system of the present disclosure in a sample, the following experiment was performed.

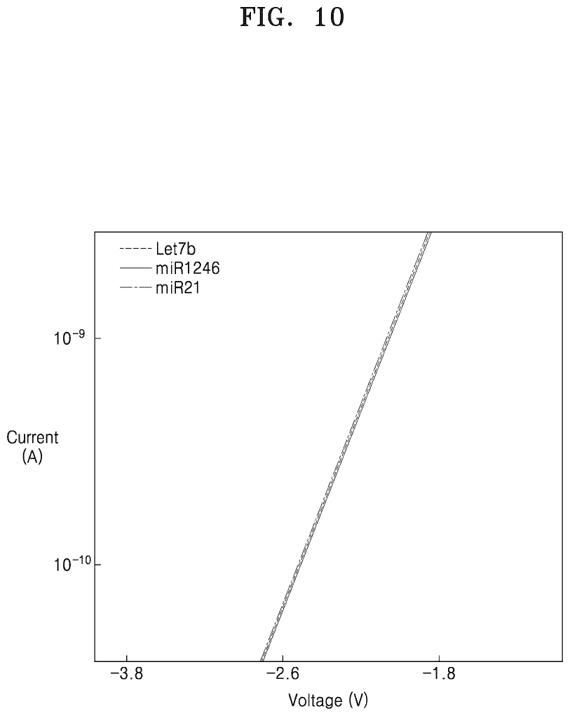

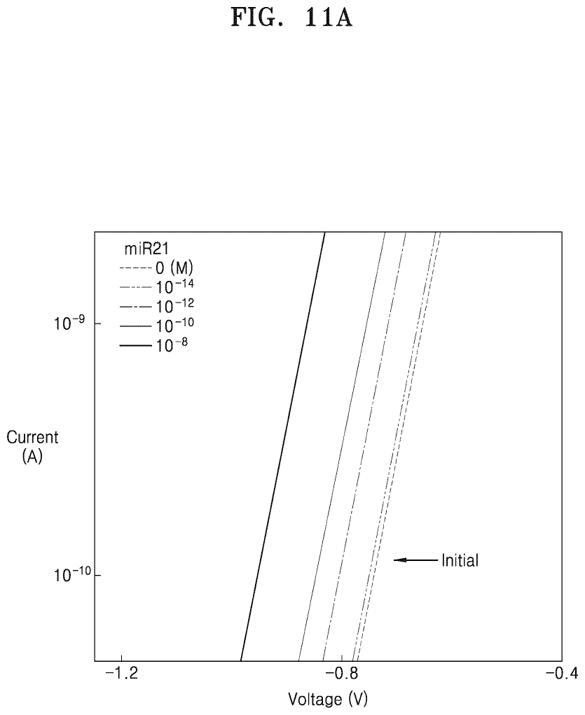

First, a probe capable of detecting Let7b, miR1246, or miR21 was immobilized in each well of the disposable sensor chip of the present disclosure, and as a result of confirming whether there was a difference in electrical signals among the wells, little difference was confirmed (see FIG. 10).

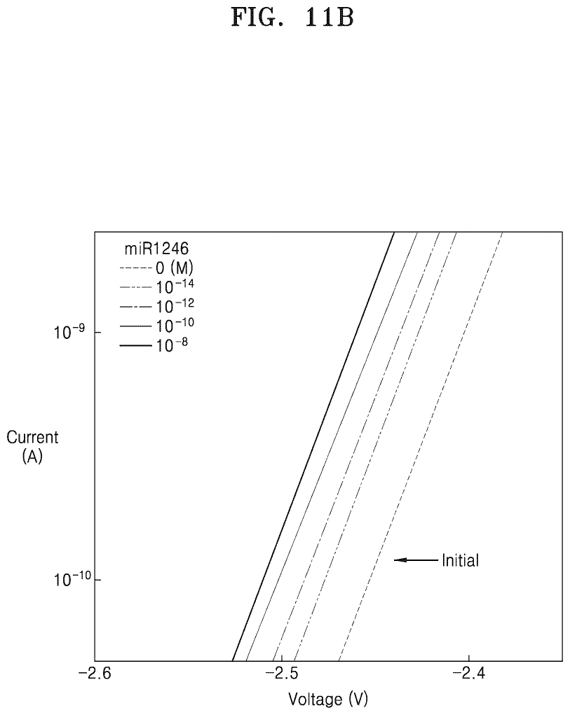

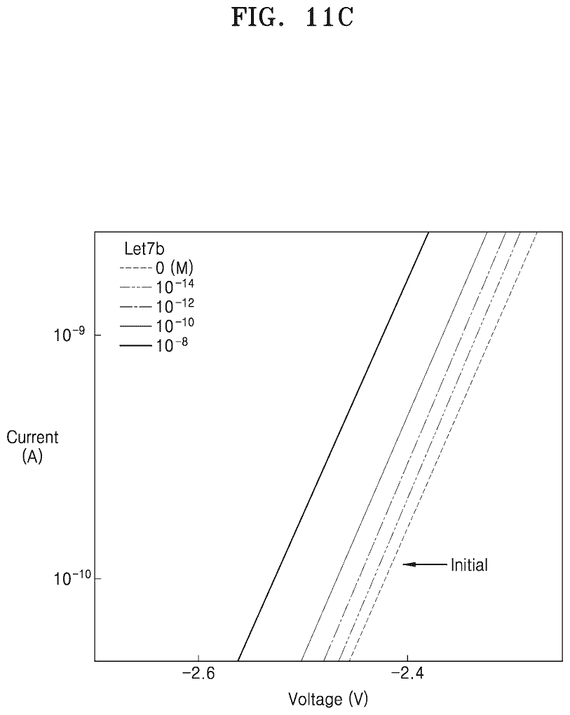

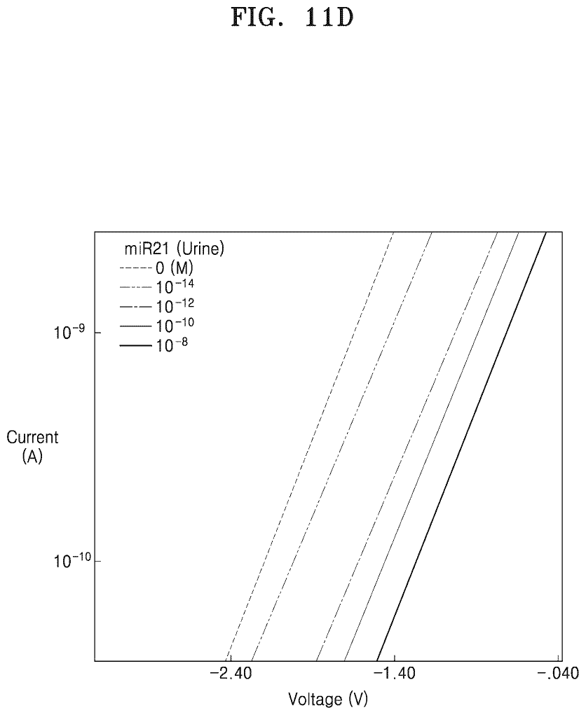

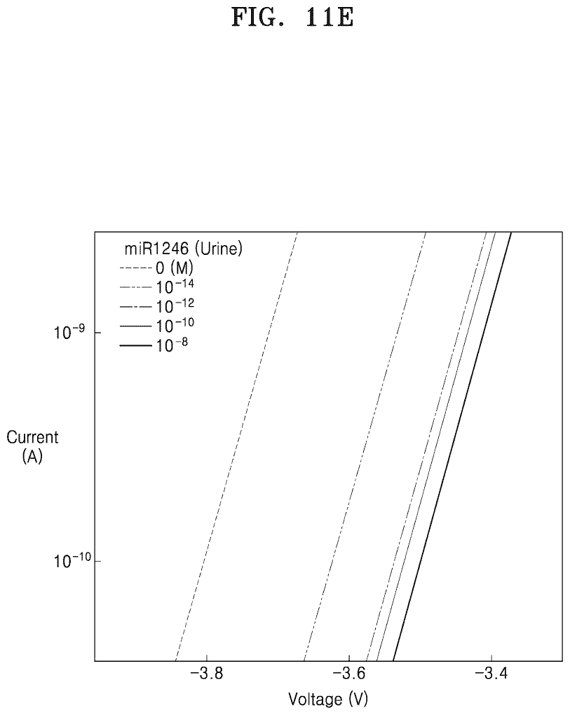

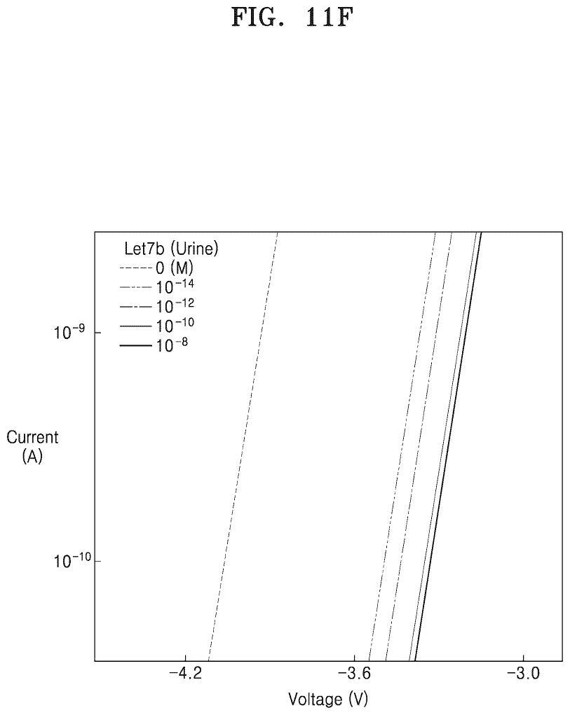

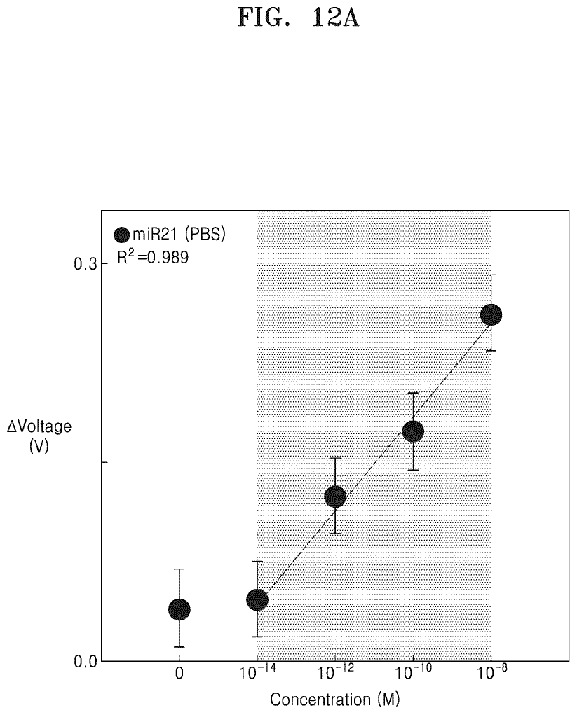

Next, to confirm the electrical signals according to the concentration of miRNA, the following experiment was performed. In detail, to obtain an I_{D}-V_{G} curve by using the FET, a dual-channel parameter analyzer (4200A-SCS, Keithley) was used. In the case of I_{D}-V_{G} curve, the sweeping voltage of the bottom gate and drain voltage ranged from −5 to 5 V, respectively. To obtain a standard curve, voltage changes according to various concentrations of miRNA were plotted, and the initial voltage value in the I_{D}-V_{G} curve was determined as a V_{G} value at 10 nA. In addition, after culturing in the miRNA solution for 20 minutes, the detection voltage was measured at 10 nA, and the final electrical signal was normalized by subtracting the initial signal of 1×PBS itself from the baseline of the endpoint signal cultured for 20 minutes. As a result, the amplification curves of the sensor showed similar trends for the three target miRNAs. In detail, it was confirmed that the I_{D}-V_{G} curve showed gradual shift in the same direction according to various miRNA concentrations in ×PBS and undiluted normal urine (see FIGS. 11A to 11F).

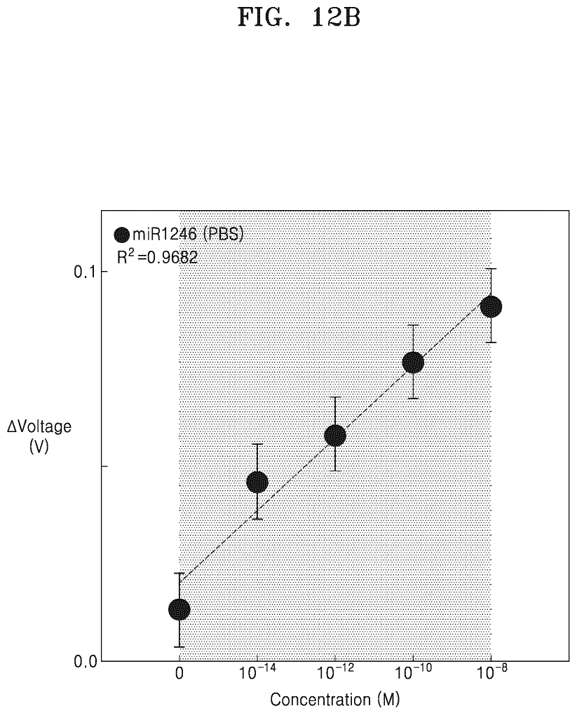

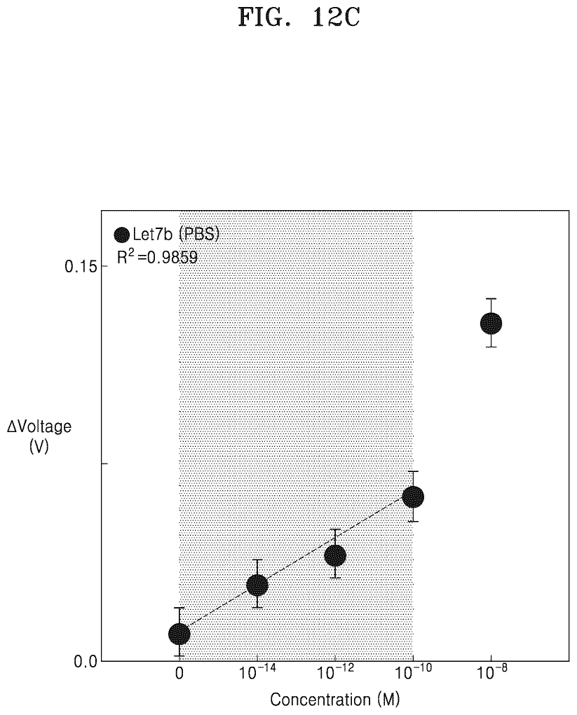

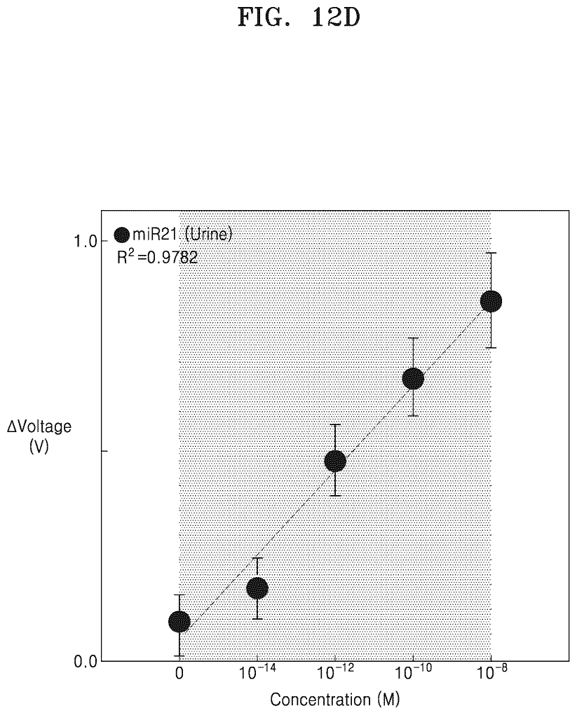

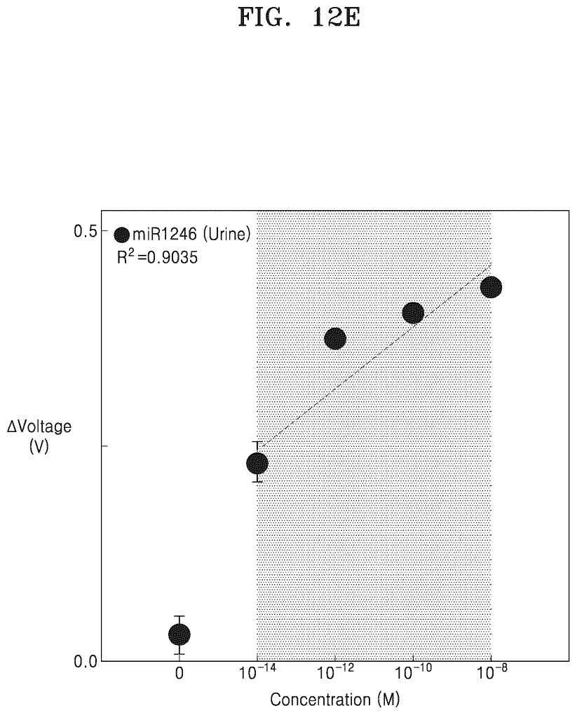

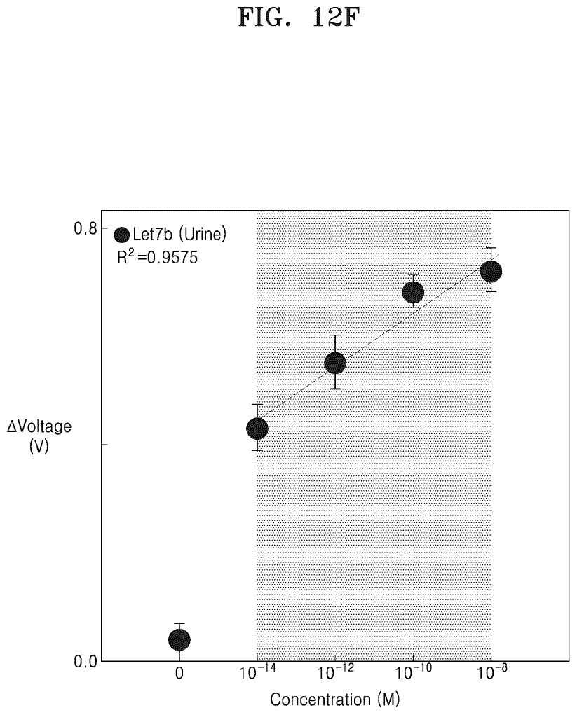

In addition, it was confirmed that the standard curve of the sensor for the three miRNAs in a concentration range of 10 fM to 10 nM exhibited a dynamic range with excellent linearity (see FIG. 12). In detail, the standard curves for miRNA21, miRNA1246, and let7b showed excellent linearity with R² of 0.9 or more in the urine samples as well as in PBS. In particular, the standard curves for miRNA21 and let7b showed much better linearity. Based on these results, it was confirmed that the miRNA sensor of the present disclosure exhibited significantly excellent analysis performance, compared with other existing analysis methods and sensors for detecting urine miRNA. In detail, the sensor of the present disclosure exhibited excellent linearity and a low limit of detection (LOD) of 10 fM, thereby confirming that the sensor was available for quantitative analysis of a wide range of biomarker concentrations.

### Example 5: Clinical Evaluation of miRNA Sensor

To confirm whether the miRNA sensor of the present disclosure was able to effectively detect target miRNA to be detected in an actual urine sample of a patient, the following experiment was performed.

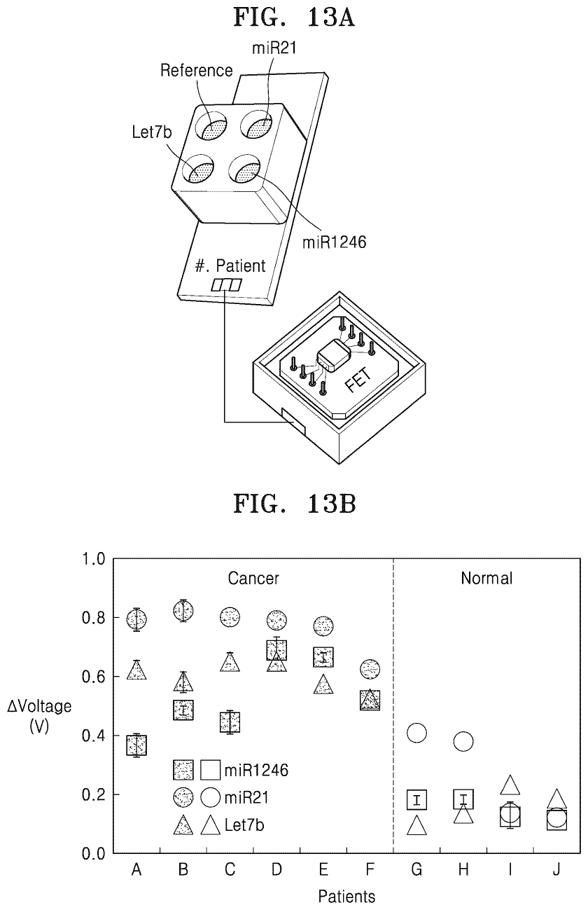

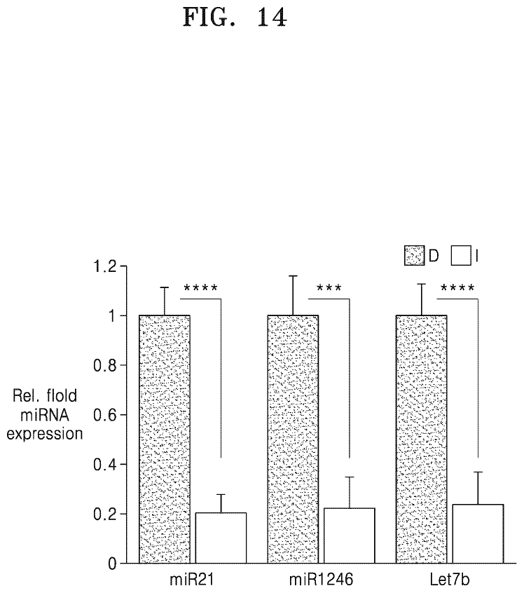

First, 10 urine samples of intact human patients and normal people were prepared without pretreatment. Then, the electrical signal shift of ID-VG was measured in the same manner as in Examples above. Consequently, for miRNA21, miRNA1246, and let7b, a significant difference in voltage signal fluctuations between the patients with prostate cancer and normal people was shown (see FIGS. 13A to 13B), high levels of cancer-specific miRNAs were detected in the patients with cancer (Patents A to F in Table 2) compared to normal people, and the electrical signal values corresponding to each miRNA were found to be within the linear range of the standard curve. In particular, it was confirmed that, compared to other miRNAs, let7b exhibited a significantly great difference in the voltage signal fluctuations between the patients with prostate cancer and normal people. The results thus obtained were also confirmed through the pathological diagnosis of the patients described in Table 2 and the miRNA expression profile by real-time qPCR (see FIG. 14).

TABLE 2

| | | PSA level | Voltage change ΔV (mV) | | | Pathological | | Cellular |
|---|---|---|---|---|---|---|---|---|
| Patient | Age | (ng/mL) | miR21 | miR1246 | Let7b | state | Grade | state |
| A | 69 | 4.99 | 789 | 365 | 626 | Adenocarcinoma | GS6 | Negative |
| | | | | | | (Adenocarcinoma) | (3 + 3) | (negative) |
| B | 80 | 9.84 | 820 | 483 | 576 | Adenocarcinoma | GS6 | Negative |
| | | | | | | | (3 + 3) | |
| C | 58 | 97.25 | 797 | 444 | 680 | Adenocarcinoma | GS8 | Negative |
| | | | | | | | (4 + 4) | |
| D | 80 | 7.93 | 785 | 824 | 656 | Adenocarcinoma | GS8 | — |
| | | | | | | | (4 + 4) | |
| E | 56 | 8.03 | 761 | 661 | 574 | Adenocarcinoma | GS9 | Negative |
| | | | | | | | (4 + 5) | |
| F | 69 | 6.85 | 617 | 517 | 523 | Adenocarcinoma | GS8 | Negative |
| | | | | | | | (4 + 4) | |
| G | 77 | 0.845 | 406 | 179 | 103 | Normal | — | — |
| H | 62 | 5.87 | 375 | 183 | 137 | Normal | — | — |
| I | 62 | 3.13 | 133 | 123 | 230 | Normal | — | — |
| J | 78 | 6.09 | 119 | 109 | 187 | Normal | — | — |

Furthermore, as shown in Table 2, Patients H and J who are normal people had higher prostate-specific antigen (PSA) levels compared to Patient A. Such a result indicates that the initial diagnosis result of prostate cancer before performing biopsy was different from the actual result. The PSA level in serum is known as the most important reference for the primary diagnosis of prostate cancer. However, since irregular expression patterns of PSA in patients may appear as described above, there is a need to be supplemented by other additional detection methods. In this regard, the results thus obtained indicate that the miRNA sensor of the present disclosure has potential to supplement the accuracy of the standard PSA test for early diagnosis of prostate cancer.

Overall, the present disclosure developed a label-free urine-miRNA sensing system including a disposable and switchable graphene-based electrical sensor that can simply connect to a sensing module (reaction unit) to the FET body. The mlRNA sensing system can detect miRNA in a patient urine sample in real time without pre-treatment or signal amplification steps, thereby providing a reliable and practical method. The monitoring technology of the sensor exhibits fast response time, durability, stability, and improved specificity and sensitivity. Based on the advantages resulting from the surface modification of the disposable sensor chip using biocompatible graphene nanosheet and the subsequent immobilization of the PNA probe, the performance of the sensor retains the reliability of the limit of detection (LOD) to the concentration below the subfemtomolar level of miRNA both in the 1×PBS solution and the human urine sample within 20 minutes. In addition, in a non-invasive point-of-care diagnostic system, detaching the disposable sensor chip from the FET body provides a highly durable and reusable transducer while maintaining sensing performance, and also offers the potential to create an extensible platform for the detection of various miRNAs. Finally, the sensor provides a complementary strategy to improve the accuracy of tests for early diagnosis of prostate cancer, thereby combining standard serum PSA and urine miRNA tests prior to performing a biopsy. As such, the miRNA-sensing system of the present disclosure is a promising device for field use in biomedical applications.

The foregoing descriptions are only for illustrating the present disclosure, and it will be apparent to a person having ordinary skill in the art to which the present invention pertains that the embodiments disclosed herein can be easily modified into other specific forms without changing the technical spirit or essential features. Therefore, it should be understood that Examples described herein are illustrative in all respects and are not limited.

### EXPLANATION OF REFERENCE NUMERALS

110: Reaction unit
111: Substrate
112: Working electrode
113: Insulating electrode
114: Reduced graphene oxide nanosheet
115: Test cell
116: Reference electrode

1. A biosensor comprising a reaction unit in which an interaction occurs physically or chemically with a target material in a sample, wherein
the reaction unit comprises reduced graphene oxide (rGO).

2. The biosensor of claim 1, wherein the reaction unit comprises: a substrate; an electrode formed on the substrate; and an rGO layer formed on the electrode.

3. The biosensor of claim 1, wherein the reaction unit comprises a biological probe that specifically binds to the target material.

4. The biosensor of claim 3, wherein the biological probe comprises one or more selected from the group consisting of DNA, RNA, PNA, a nucleotide, a nucleoside, a protein, a polypeptide, a peptide, an amino acid, a carbohydrate, an enzyme, an antibody, an antigen, a receptor, a virus, a substrate, a ligand, and a membrane.

5. The biosensor of claim 3, wherein the biological probe is for detecting one or more miRNAs selected from the group consisting of miRNA21, miRNA1246, and let7b.

6. The biosensor of claim 3, wherein the reaction unit comprises a linker to immobilize the biological probe on a surface of the electrode.

7. The biosensor of claim 6, wherein the linker comprises one or more selected from the group consisting of compounds comprising biotin, avidin, streptavidin, carbohydrate, poly L-lysine, a hydroxyl group, a thiol group, an amine group, an alcohol group, a carboxyl group, an amino group, a sulfur group, an aldehyde group, a carbonyl group, a succinimide group, a maleimide group, an epoxy group, and an isothiocyanate group.

8. The biosensor of claim 1, wherein the target material comprises one or more selected from the group consisting of an antigen, a haptene, a carbohydrate, a protein, a drug, a microorganism, an antibody, and a nucleic acid capable of participating in a sequence-specific hybridization reaction with a complementary sequence.

9. The biosensor of claim 1, wherein the biosensor is capable of detecting the target material in a urine sample.

10. The biosensor of claim 1, wherein the target material is a prostate cancer marker.

11. The biosensor of claim 1, wherein the biosensor further comprises a signal converter that recognizes and converts the interaction into an electrical signal, and the reaction unit is separable from the signal converter.

12. The biosensor of claim 11, wherein the signal converter comprises a field-effect transistor (FET).

13. The biosensor of claim 12, wherein the FET comprises: a substrate; an insulating layer; a source electrode and a drain electrode that are spaced apart from each other; a gate electrode; and a channel layer arranged between the source electrode and the drain electrode.

14. A method of detecting a target material, the method comprising: contacting a sample to the biosensor of claim 1; and observing a change in electrical conductivity of the biosensor.

15. The method of claim 14, wherein the reaction unit comprises a biological probe that specifically binds to the target material; and wherein the biological probe is for detecting one or more miRNAs selected from the group consisting of miRNA21, miRNA1246, and let7b.

16. The method of claim 14, wherein the biosensor is capable of detecting the target material in a urine sample.

17. The method of claim 14, wherein the target material is a prostate cancer marker.

18. A method of providing information on the diagnosis of prostate cancer, the method comprising: measuring an expression level of a prostate cancer marker by using the biosensor of claim 1, from a biological sample isolated from a subject.

19. The method of claim 18, wherein the prostate cancer marker comprise one or more selected from the group consisting of miRNA21, miRNA1246, and let7b.


SEQUENCE LISTING (7 sequences)

SEQ ID NO: 1 - DNA, 22 bp, Artificial
  PNA-21
tcaacatcagtctgataagcta

SEQ ID NO: 2 - DNA, 19 bp, Artificial
  PNA-1246
cctgctccaaaaatccatt

SEQ ID NO: 3 - DNA, 22 bp, Artificial
  PNA-let7b
aaccacacaacctactacctca

SEQ ID NO: 4 - RNA, 22 nt, Artificial
  miRNA21
uagcuuaucagacugauguuga

SEQ ID NO: 5 - RNA, 19 nt, Artificial
  miRNA1246
aauggauuuuuggagcagg

SEQ ID NO: 6 - RNA, 22 nt, Artificial
  let7b
ugagguaguagguugugugguu

SEQ ID NO: 7 - RNA, 22 nt, Artificial
  mismatch
uagcuuaucauacugauguuga